(19) United States
(12) Patent Application Publication
(10) Pub. No.: US 2011/0244222 A1
(43) Pub. Date: Oct. 6, 2011

(54) SEAMLESS BELT

(75) Inventors: Ji Sung KIM, Seoul (KR); Ki Nam KWAK, Gyeonggi-do (KR); Jeong Han KIM, Gyeonggi-do (KR)

(73) Assignee: KOLON INDUSTRIES, INC., Gwacheon-si (KR)

(21) Appl. No.: 13/074,489

(22) Filed: Mar. 29, 2011

(30) Foreign Application Priority Data
Mar. 30, 2010 (KR) 10-2010-0028372

(51) Int. Cl. G03G 15/14; G03G 15/20; C07C 11/00; C07C 69/74; C09D 191/00; B32B 9/00

(52) U.S. Cl. 428/336; 399/313; 399/329; 585/16; 560/1; 106/271

(57) ABSTRACT

The present invention relates to a belt for an image forming apparatus, including a releasing coating layer containing at least one selected from the group consisting of a paraffin-based hydrocarbon that is also known as a paraffin wax, an isomer thereof or ester compound.

### TECHNICAL FIELD

The present invention relates to a seamless belt that can be used in a fixing belt, an intermediate transfer belt, and the like of an image forming apparatus.

### BACKGROUND ART

For an image forming apparatus that is used in the image forming apparatus, such as a copy machine, a facsimile, a printer, and the like, generally a trommel of metallic material or a trommel of resin material that is composed of a polyimide resin is in use as a fixing belt for the necessity of high density driving in a belt for the image forming apparatus, such as a fixing belt that is used in a fixing part, an intermediate transfer belt that is used in a transfer part, for example.

The fixing belt requires a thermal resistance about a fixing temperature, or a releasability about a toner. For this reason, an external side (i.e., the side that is contacted to a toner) of the fixing belt is covered with a releasing film having a thermal resistance, such as a fluoroplastic, and the like. In addition, the fixing belt requires a durability that can withstand copying of about 100,000 pages at a rate of 30-40 pages/minutes. For this reason, the fixing belt requires a strong adhesive property between a material layer and a releasing layer.

The above fixing belt may be generally obtained from a method comprising: molding and drying a trommel from a solution of polyimide precursor that is obtained by reacting a diamine and a tetracarboxylic acid dianhydride in a polar polymerization solvent; applying a primer on the above trommel, and drying; applying a fluoroplastic dispersion on the above primer layer, and drying; and finally politicizing a fluoroplastic, along with imidizing a polyimide precursor under a high temperature; or a method comprising: processing a metallic material into a trommel; applying a primer on the above trommel, and drying; applying a fluoroplastic dispersion on the above primer layer, and drying; and politicizing; and the like. For a method for preparing the trommel from the solution of the polyimide precursor, for example it has been proposed to obtain the polyimide trommel by separating from a mold by chemically imidizing or heating after molding the solution of the polymide precursor in a fixed thickness on an external side or an internal side of a mold for molding.

However, in the case of the fluoroplastic that is a main material of resin layer having the releasability, the modification temperature is about 280° C. so that there may be limit to not exceed 280° C. of a continuous use temperature for the transfer belt or the fixing belt including the above resin layer having the releasability.

### DISCLOSURE

### Technical Problem

An embodiment according to the present invention provides a seamless belt including a releasing layer having an improved thermal resistance.

An embodiment according to the present invention provides a seamless belt including a releasing coating layer including at least one selected from the group consisting of a paraffin-based hydrocarbon represented by the following Chemical Formula 1 and Chemical Formula 2, an isomer thereof or ester compound.

CₙH₂ₙ₊₂ [Chemical Formula 1]

In the above Chemical Formula 1, n is an integer of not less than 20.

RCOOR′ [Chemical Formula 2]

In the above Chemical Formula 2, R is CₘH₂ₘ₊₁, R′ is CₚH₂ₚ₊₁, m and p are an integer, respectively, and m+p that is a sum thereof is an integer of not less than 20.

Considering the improvement of the continuous use temperature of the seamless belt according to an embodiment of the present invention, at least one selected from the group consisting of the paraffin-based hydrocarbon represented by Chemical Formula 1 and Chemical Formula 2, the isomer thereof or ester compound, which is included in the releasing coating layer, may preferably have at least 300° C. of the boiling point.

In addition, for the seamless belt according to an embodiment of the present invention, the paraffin-based hydrocarbon or isomer thereof has a limitation on their purification so that a carbon number in the above Chemical Formula 1 and Chemical Formula 2 is difficult to have a specific number. Thus, preferably, “n” is preferably not less than 20 or “m+p” is not less than 20, and the above compounds are preferably included in at least 50 wt % based on weight of the releasing coating layer in terms of the satisfaction of the releasability, an antifouling, the thermal resistance, and a surface property.

The releasing coating layer is preferably composed of naphtha, solvent Naphtha, carbon tetrachloride, benzene, chloroform, dichloromethane, dichloroethane, ligroin, petroleum ether, isobutylether, ether, pentane, hexane, heptane, octane, isodecane, and the like as a solvent in terms of solubility, and also alcohol (not less than 95% of purity), acetone, ethyl acetate, and the like may be possible to apply a thin film coating, but they have very low solubility so that their usages are not preferable.

In addition, the paraffin-based hydrocarbon that is also known as paraffin wax, the isomer thereof, or ester compound have low compatibility with a polymer component, such as polyimide, polyamide, and the like that compose the intermediate transfer belt or the fixing belt so that they may be not proper to mix and then use, but they may be preferably used for the external side of the seamless belt in terms of the expression of the releasability.

There is no reason to specifically limit a thickness of the releasing coating layer in the case of the fixing belt. However if it is formed in the thin film, it has a to sufficient releasability and antifouling so that the thickness may be preferably not more than 25.0 μm. Also, in the case of the intermediate transfer belt, if the thickness is thick, the surface resistance property that is required for the intermediate transfer belt may be distorted so that the thickness may be preferably about 0.05 to 3.0 μm.

The seamless belt according to the present invention may be used as the intermediate transfer belt of the image forming apparatus.

The seamless belt according to the present invention may be used as the fixing belt of the image forming apparatus.

An embodiment of the present invention may provide the seamless belt having the releasing coating layer having the thermal resistance that has at least 300° C. of a continuous use temperature, the antifouling, or an excellent adhesive property to a substrate, and the seamless belt obtained from the above can be used as the intermediate transfer belt or the fixing belt having the thermal resistance.

### Technical Solution

The present invention will be described in more detail as follows.

A belt for an image forming apparatus according to an embodiment of the present invention includes a releasing coating layer including at least one selected from the group consisting of a paraffin-based hydrocarbon that is also known as a paraffin wax and represented by the following Chemical Formula 1 and Chemical Formula 2, an isomer thereof or ester compound.

CₙH₂ₙ₊₂ [Chemical Formula 1]

In the above Chemical Formula 1, n is an integer of not less than 20.

RCOOR′ [Chemical Formula 2]

In the above Chemical Formula 2, R is CₘH₂ₘ₊₁, R′ is CₚH₂ₚ₊₁, m and p are an integer, respectively, and m+p that is a sum thereof is an integer of not less than 20.

Example of the paraffin wax represented by Chemical Formula 1 may be the component as the following Table 1, but it is not limited to normal paraffin.

TABLE 1

| | | | Melt- | Boil- | |
|---|---|---|---|---|---|
| | | | ing | ing | |
| Value | | Chemical | Point | Point | |
| of n | Chemical Name | Formula | (° C.) | (° C.) | CAS RN |
| 20 | n-Eicosane | C20H42(282.55) | 37 | 343 | 112-95-8 |
| 21 | n-Henicosane | C21H44(296.58) | 40.5 | 356.5 | 629-94-7 |
| 22 | n-Docosane | C22H46(310.61) | 44.5 | 368.5 | 629-97-0 |
| 23 | n-Tricosane | C23H48(324.63) | 47.5 | 380 | 638-67-5 |
| 24 | n-Tetracosane | C24H50(338.66) | 52 | 391 | 646-31-1 |
| 25 | n-Pentacosane | C25H52(352.69) | 54 | 402 | 629-99-2 |
| 26 | n-Hexacosane | C26H54(366.71) | 56.5 | 412 | 630-01-3 |
| 27 | n-Heptacosane | C27H56(380.74) | 59 | — | 593-49-7 |
| 28 | n-Octacosane | C28H58(394.77) | 64.5 | 431.5 | 630-02-4 |
| 29 | n-Nonacosane | C29H60(408.79) | 65 | — | 630-03-5 |
| 30 | n-Triacontane | C30H62(422.82) | 66 | 450 | 638-68-6 |
| 31 | n-Hentriacontane | C31H64(436.85) | 67 | — | 630-04-6 |
| 32 | n-Dotriacontane | C32H66(450.87) | 69 | 467 | 544-85-4 |
| 33 | n-Triatriacontane | C33H68(464.90) | 71 | — | 630-05-7 |
| 34 | n-Tetratriacontane | C34H70(478.93) | 72 | — | 14167- |
| | | | | | 59-0 |
| 35 | n-Pentatriacontane | C35H72(492.95) | 73 | — | 630-07-9 |
| 36 | n-Hexatriacontane | C36H74(506.98) | 74 | — | 630-06-8 |
| 37 | n-Heptatriacontane | C37H76(521.01) | 76 | — | 7194-84-5 |
| 38 | n-Octatriacontane | C38H78(535.03) | 78 | — | 7194-85-6 |
| 39 | n-Nonatriacontane | C39H80(549.05) | 80 | — | 7194-86-7 |
| 40 | n-Tetracontane | C40H82(563.09) | 82 | — | 4181-95-7 |
| 42 | n-Dotetracontane | C42H86(591.13) | 84 | — | 7098-20-6 |
| 44 | n-Tetratetracontane | C44H90(619.20) | 85 | — | 7098-22-8 |
| 46 | n-Hexatetracontane | C46H94(647.24) | 86 | — | 7098-24-0 |
| 48 | n-Octatetracontane | C48H98(675.30) | 90 | — | 7098-26-2 |
| 50 | n-Pentacontane | C50H102(703.36) | 91 | — | 6596-40-3 |
| 52 | n-Dopentacontane | C52H106(731.40) | 92 | — | 7719-79-1 |
| 54 | n-Tetrapentacontane | C54H110(759.45) | 93 | — | 5856-66-6 |
| 56 | n-Hexapentacontane | C56H114(787.50) | 94 | — | 7719-82-6 |
| 58 | n-Octapentacontane | C58H118(815.56) | 95 | — | 7667-78-9 |
| 60 | n-Hexacontane | C60H122(843.62) | 96 | — | 7667-80-3 |
| 70 | n-Heptacontane | C70H142 | 105.5 | — | |

As shown in the above Table 1, it could be known that when the value of n is at least 20 and the boiling point is not less than 300° C., as the value of n is increased, the boiling point is also increased. For the above Table 1, it could be understood that when some compounds do not have the value of the boiling point, the values cannot be easily determined or the boiling point cannot be observed for the compounds that are industrially produced. Thus, it has sufficiently meanings to determine the change of the boiling point according to the value of n. Meanwhile, example of the compound represented by Chemical Formula 2 may include a hexadecyl palmitate, in which m is 15 and p is 16 as the following a structural formula:

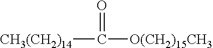

(Melting point: 53° C., Boiling point: 534° C.)

In addition, it may include a methyl behenate, in which m is 21 and p is 1 as the following structural formula:

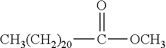

(Melting point: 55° C., Boiling point: 393° C.)

The above two compounds are given as an example, but the compound represented by Chemical Formula 2 disclosed in the present invention is not limited thereto.

The paraffin-based hydrocarbon represented by Chemical Formula 1 may be an isoparaffin-based hydrocarbon, or a synthetic isoparaffin-based hydrocarbon. The compound represented by Chemical Formula 2 is ester compound of a linear fatty acid. The paraffin-based hydrocarbon that is also known as the paraffin wax, the isomer thereof, or ester compound exhibits an adhesive strength about the substrate when preparing a film and also its surface has the releasability at the same time so that it can exhibit the releasability about the toner when locating on a outmost surface of the seamless belt. At this time, the substrate may be a metallic material, or a polycarbonate-based resin or a polyimide-based resin, and the adhesive strengths about each substrate are the same.

For an organic material represented by the above Chemical Formula 1, n should be at least 18 so that the stable solid state can be maintained at a room temperature, i.e., 25° C. The present invention makes n possible to be at least 20 thereby maintaining the stable solid state at not less than 30° C. and preferably maintaining the stable solid state at not less than 100° C.

In addition, for this reason, the boiling point can be maintained at not less than 300° C. so that the releasing coating layer having the thermal resistance can be formed.

The paraffin-based hydrocarbon disclosed in the present invention is obtained by removing the hydrocarbon having low carbon number (hereinafter, called as a lower hydrocarbon that has not less than 20 of carbon number) through a purification or distillation from the paraffin-based hydrocarbon mixture that is obtained from a petroleum resin or the synthetic paraffin-based hydrocarbon mixture, and the paraffin-based hydrocarbon without the lower hydrocarbon (hereinafter, called as a higher hydrocarbon that has above 20 of carbon number) includes the hydrocarbon, of which the carbon number and the molecular structure are exactly unknown, as well as the hydrocarbon, of which the carbon number is exactly known. In addition, the higher hydrocarbon mixture is composed of many isoparaffin-based hydrocarbons and also the normal paraffin-based hydrocarbon. The releasing coating layer according to the present invention is preferably composed of the higher hydrocarbon, and more preferably is applied with the residual hydrocarbon mixture that is remained from removing the higher hydrocarbon having 20 to 70 of carbon number from the higher hydrocarbon mixture through a vacuum distillation. The above residual hydrocarbon mixture has not less than 100° C. of the melting point and also the boiling point having a high level that is exactly unknown. However, the higher hydrocarbon is produced by continuously repeating the distillation processes or using extreme conditions, such as high temperature/low pressure so that they cause the cost increase in economic term. Therefore, the application of the paraffin-based hydrocarbon mixture that is composed of not less than 20 carbon number is the most economical.

Therefore, the releasing coating layer may be composed of only one component selected from the group consisting of the paraffin-based hydrocarbon that is also known as the paraffin wax, the isomer thereof or ester compound, and may be mixed with at least two components. At least two of the paraffin-based hydrocarbon that is also known as the paraffin wax, the isomer thereof, or ester compound have a slightly difference in terms of the components and the chemical structures, but have similar levels in terms of the releasability, the antifouling and the thermal resistance.

The higher hydrocarbon is composed of the normal paraffin-based hydrocarbon or the isoparaffin-based hydrocarbon, and if the carbon number is increased, the thermal resistance becomes excellent. The higher hydrocarbon having many carbon numbers expresses very excellent thermal resistance so that the substrate including the higher hydrocarbon thin film having the thermal resistance that is required can be implemented by heating at a high temperature after coating on the substrate surface. The above heating can evaporate a hydrocarbon having a poor thermal resistance to remove thereby forming the thin film layer that is composed of only the higher hydrocarbon.

Generally, the releasing coating layer may further include other components that compose the releasing coating layer.

At this time, the content of the component represented by the above Chemical Formula 1 to Chemical Formula 2 in the releasing coating layer is at least 50 wt % and preferably 70 wt % so that the intended goal can be accomplished.

Example of other components that compose the releasing coating layer may include for example, a filler, a dye, a pigment, a pigment dispersant, a solid lubricant, an anti-settling agent, a leveling agent, a surface modifier, a moisture absorbent, an anti-gelling agent, an antioxidant, a ultraviolet absorbent, a light stabilizer, a plasitcizer, an anti-flooding agent, an anti-skinning agent, a surfactant, an anti-foamer agent, an anti-microbial agent, an anti-fungal agent, a preservative, a thickener, a thermally conductive agent, and the like as a known additional agent in the range of not affecting the purpose of the present invention.

The releasing coating layer is preferably formed from one or two or more mixture solution selected from the group consisting of naphtha, solvent Naphtha, carbon tetrachloride, benzene, chloroform, dichloromethane, dichloroethane, ligroin, petroleum ether, isobutylether, ether, pentane, hexane, heptane, octane, isodecane, and the like as a solvent in terms of solubility, and also alcohol (not less than 95% of purity), acetone, ethyl acetate, and the like has very low solubility so that they may be applied as the solution mixed with the above solvent.

A resin or metal may be used as a substrate when the seamless belt for the image forming apparatus, such as the fixing belt or transfer belt is prepared by using the releasing coating solution.

For example, there is a resin having the thermal resistance, such as a polyimide resin, a polyamideimide resin, a polyetheretherketone resin, a polyphenylenesulfide resin, a polyethersulfone resin, and the like as a resin, and can be used as the substrate of the intermediate transfer belt and the fixing belt. In addition, it can be a polycarbonate, a silicon resin, a rubber resin, and the like, and can be used as the substrate of the intermediate transfer belt. For example, silver, aluminum, nickel, iron, stainless steel, and the like can be used as the metal. In addition, one laminated with two or more of at least one side of resin and metal can be used as the substrate.

For example, the resin may include an electric conductive filler, a filler for reinforcing a mechanical property, a pigment, a pigment dispersant, a solid lubricant, an anti-settling agent, a leveling agent, a surface modifier, a moisture absorbent, an anti-gelling agent, an antioxidant, a ultraviolet absorbent, a light stabilizer, a plasitcizer, an anti-flooding agent, an anti-skinning agent, a surfactant, an antistic agent, an anti-foamer agent, an anti-microbial agent, an anti-fungal agent, a preservative, a thickener, a thermally conductive agent, and the like as a known additional agent in the range of not affecting the purpose of the present invention.

The shape of the substrate is not limited specifically, but can be a cylinder-shape, a sheet-shape, or other shapes. However, the substrate is preferably a trommel when preparing a toner fixing belt of the transfer photo image forming apparatus, such as a facsimile, a copy machine, a laser beam printer, and the like.

A method for applying the releasing coating solution on the substrate may include a known method, such as a brushing method, a spin coating method, a spraying method, a dispenser method, an immersing method, a pressing method, and the like, and the spraying method is more preferably used for the application of the intermediate transfer belt and the fixing belt.

The plasticization process can be performed after coating, and the condition for the plasticization process may be 60° C. to 200° C. The plasticization process is to provide a semi-permanent releasability, and also is not necessarily required.

The thickness of the releasing coating layer may be preferably about 0.05 to 25 μm after coating, or coating and plasticizing, in terms of the sufficient expression of the releasability. The thickness of the releasing coating layer is not limited specifically in the case of the fixing belt, but when the releasing coating layer is formed as a thin film, the sufficient releasability and anti-fouling are appeared, so that it may preferably be not more than 25.0 μm; and in the case of the intermediate transfer belt, when the thickness is thick, the surface resistance characteristic that is required for the intermediate transfer belt may be distorted so that the thickness may preferably be about 0.05 to 3.0 μm.

The releasing coating layer may preferably be located on the outmost of the belt considering the releasability with the toner.

### BEST MODE

Hereinafter, the embodiments of the present invention will be described in detail with reference to Examples as follows, but is not limited to the following Examples.

### Example 1

A hexacosane (carbon number n=26, available from Tokyo Chemical Industry, Co. Ltd) that is the paraffin-based hydrocarbon was dissolved in a normal hexane to be 5 wt % of solid content to prepare a produced solution for forming the releasing layer.

The produced solution was coated on the external sides of a stainless trammel (Inside diameter: 24 mm, Thickness: 0.1 mm) and a polyimide trammel (Inside diameter: 185 mm, Thickness: 65 μm) based on BPDA-ODA-PPDA through a spraying method; then plasitized for 20 minutes at 120° C.; and then applied to be 0.2 μm mean thickness to obtain a final cylindrical laminate.

(1) Estimation of Releasing Coating Layer

After spraying thinly a toner (Black for Samsung Electronics CLP-300) that is used for a color laser printer on the seamless belt prepared, the sprayed toner was removed by using an adhesive tape (available from 3M Company, Scotch Magic Tape). At this time, it was confirmed whether the toner was left or not.

(2) Estimation as Seamless Belt for Copy Machine

After producing the seamless belt as the intermediate transfer belt or the fixing belt of a color laser printer (Samsung Electronics CLP-300), it was replaced with the existed fixed belt and then the qualities of printed images were determined with the naked eyes before and after replacing.

(3) Estimation of Continuous Use Temperature

The fixing belt requires not less than 250° C. of a continuous use temperature. Therefore, for the estimation as the seamless belt for a copy machine as disclosed in the above (2), after applying the prepared seamless belt as the fixing belt, the qualities of the printed images of the initial printed matter and the final printed matter were compared with the naked eyes after copying 1000 pages. After only the seamless belt having a good result of the observation with the naked eyes was heated for 1 hour at 350° C. and 450° C. by using a calcinator, the printed matter was estimated and the continuous use temperature was finally estimated.

The results from the above estimations were shown in the following Table 2.

### Example 2

A paraffin wax mixture (Microcrystalline wax, Melting point: 65.5-76.6° C., Carbon number n=29 to 37, Daemyung Chemical Company, Korea) was dissolved in a isobutylether to be 5 wt % of solid content to prepare a produced solution for forming the releasing layer.

The produced solution for forming the releasing layer was applied on each trommel and plasticized to prepare a cylindrical laminate having the releasing coating layer by using the same method to the above Example 1.

It was estimated with the same method to the above Example 1, and then the result was shown in the following Table 2.

### Example 3

A paraffin wax mixture (Microcrystalline wax, Melting point: 101° C., Carbon number n=60 to 70, Nippon Seiro Company, Japan) was dissolved in a solvent naphtha and naphtha to be 3 wt % of solid content to prepare a produced solution for forming the releasing layer.

The produced solution for forming the releasing layer was applied on each trommel and plasticized to prepare a cylindrical laminate having the releasing coating layer by using the same method to the above Example 1.

It was estimated with the same method to the above Example 1, and then the result was shown in the following Table 2.

### Example 4

A hexacosane was dissolved in a isobutylether to be 5 wt % and then a methyl behenate (Melting point: 55° C., Boiling point: 393° C., Tokyo Chemical Industry Co. Ltd) that is the ester compound was dissolved to be 5 wt % so that a produced solution for forming a releasing layer having 10 wt % of total solid content.

The produced solution for forming the releasing layer was applied on each trommel and plasticized to prepare a cylindrical laminate having the releasing coating layer by using the same method to the above Example 1.

It was estimated with the same method to the above Example 1, and then the result was shown in the following Table 2.

### Example 5 and Example 6

The cylindrical laminate was prepared with the same method to the above Example 3 except 10 wt % of solid content in a produced solution for forming a releasing layer and different thickness of the releasing coating layer.

It was estimated with the same method to the above Example 1, and then the result was shown in the following Table 2.

### Example 7 to Example 10

The produced solution obtained by using the same method to the above Example 3 except 10 wt % of solid content in the produced solution for forming a releasing layer was coated on the external sides of a stainless trammel (Inside diameter: 24 mm, Thickness: 0.1 mm) and a polyimide trammel (Inside diameter: 24 mm, Thickness: 65 μm) through a spraying coating method; then plasitized for to 20 minutes at 120° C.; and then applied to be 5 to 25 μm mean thickness to obtain a final cylindrical laminate.

It was estimated with the same method to the above Example 1, and then the result was shown in the following Table 2.

TABLE 2

| | | | Thickness of | | | |
|---|---|---|---|---|---|---|
| | | | Releasing | | | Continuous |
| | | | Coating | Toner | Image | Use |
| | Substrate | Use | Layer (μm) | Releasability | Property | Temperature (° C.) |
| Example 1 | Polyimide | Intermediate | 0.2 | good | good | No Test |
| | | Transfer Belt | | | | |
| | Stainless | Fixing Belt | 0.2 | good | good | Not less than 350 |
| Example 2 | Polyimide | Intermediate | 0.2 | good | good | No Test |
| | | Transfer Belt | | | | |
| | Stainless | Fixing Belt | 0.2 | good | good | Not less than 350 |
| Example 3 | Polyimide | Intermediate | 0.2 | good | good | No Test |
| | | Transfer Belt | | | | |
| | Stainless | Fixing Belt | 0.2 | good | good | Not less than 450 |
| Example 4 | Polyimide | Intermediate | 0.2 | good | good | No Test |
| | | Transfer Belt | | | | |
| | Stainless | Fixing Belt | 0.2 | good | good | Not less than 450 |
| Example 5 | Polyimide | Intermediate | 1.0 | good | good | No Test |
| | | Transfer Belt | | | | |
| | Stainless | Fixing Belt | 1.0 | good | good | Not less than 450 |
| Example 6 | Polyimide | Intermediate | 3.0 | good | good | No Test |
| | | Transfer Belt | | | | |
| | Stainless | Fixing Belt | 3.0 | good | good | Not less than 450 |
| Example 7 | Polyimide | Fixing Belt | 5.0 | good | good | Not less than 450 |
| | Stainless | Fixing Belt | 5.0 | good | good | Not less than 450 |
| Example 8 | Polyimide | Fixing Belt | 10.0 | good | good | Not less than 450 |
| | Stainless | Fixing Belt | 10.0 | good | good | Not less than 450 |
| Example 9 | Polyimide | Fixing Belt | 15.0 | good | good | Not less than 450 |
| | Stainless | Fixing Belt | 15.0 | good | good | Not less than 450 |
| Example 10 | Polyimide | Fixing Belt | 25.0 | good | good | Not less than 450 |
| | Stainless | Fixing Belt | 25.0 | good | good | Not less than 450 |

For the above Table 2, “No Test” for a continuous use temperature could be understood that the seamless belts of the polyimide substrates that were obtained from Example 1 to Example 6 were considered as the use of the intermediate transfer belt, and generally the intermediate transfer belt is used at a room temperature so that the test was useless and the test was not performed.

From the results of the above Table 2, it could be known that the intermediate transfer belt or the fixing belt including the releasing coating layer containing the paraffin-based hydrocarbon that is also known as the paraffin wax, the isomer thereof or ester compound have an excellent adhesive property to various substrates and an excellent releasability about a toner thereby having an excellent image property. Especially, when using as the fixing belt, the continuous use temperature is increased so that the durability can substantially be improved.

1. A seamless belt including a releasing coating layer, comprising at least one selected from the group consisting of a paraffin-based hydrocarbon represented by the following Chemical Formula 1 to Chemical Formula 2, an isomer thereof or ester compound:
CₙH₂ₙ₊₂ [Chemical Formula 1]
[In the above Chemical Formula 1, n is an integer of not less than 20]
RCOOR′ [Chemical Formula 2]
[In the above Chemical Formula 2, R is CₘH₂ₘ₊₁, R′ is CₚH₂ₚ₊₁, m and p are an integer, respectively, and m+p that is a sum thereof is an integer of not less than 20]

2. The seamless belt according to claim 1, wherein at least one selected from the group consisting of the paraffin-based hydrocarbon represented by Chemical Formula 1 to Chemical Formula 2, the isomer thereof or ester compound has at least 300° C. of boiling point.

3. The seamless belt according to claim 1, wherein at least one selected from the group consisting of the paraffin-based hydrocarbon represented by Chemical Formula 1 to Chemical Formula 2, the isomer thereof or ester compound is included in at least 50 wt % based on the weight of the releasing coating layer.

4. The seamless belt according to claim 1, wherein the releasing coating layer is formed from the solution containing one or two or more mixture solution selected from the group consisting of naphtha, solvent Naphtha, carbon tetrachloride, benzene, chloroform, dichloromethane, dichloroethane, ligroin, petroleum ether, isobutylether, ether, pentane, hexane, heptane, octane, isodecane, and the like as a solvent.

5. The seamless belt according to claim 1, wherein the thickness of the releasing coating layer is 0.05 to 25 μm.

6. The seamless belt according to claim 1, wherein the thickness of the releasing coating layer is 0.05 to 3.0 μm.

7. The seamless belt according to claim 1, wherein the releasing coating layer is formed on an outmost surface.

8. The seamless belt according to claim 1, wherein the seamless belt is an intermediate transfer belt of an image forming apparatus.

9. The seamless belt according to claim 1, wherein the seamless belt is a fixing belt of an image forming apparatus.
